(19) United States
(12) Patent Application Publication
(10) Pub. No.: US 2025/0255811 A1
(43) Pub. Date: Aug. 14, 2025

(54) LOW ENERGY CONSUMPTION CONTINUOUS METHOD FOR THE PRODUCTION OF SUV, LUV AND GUV UNILAMELLAR LIPOSOMES

(71) Applicants: UNIVERSITA' DEGLI STUDI DI ROMA "LA SAPIENZA" (60%), Roma (IT); THE FIRST HOSPITAL OF CHINA MEDICAL UNIVERSITY (40%), Roma (IT)

(72) Inventors: Marco Stoller, Roma (IT); Xinghua Gao, Roma (IT); Ruiqun Qi, Roma (IT); Li Zhang, Roma (IT); Xuegang Xu, Roma (IT); Giorgio Vilardi, Roma (IT)

(73) Assignees: UNIVERSITA' DEGLI STUDI DI ROMA "LA SAPIENZA" (60/%), Roma (IT); THE FIRST HOSPITAL OF CHINA MEDICAL UNIVERSITY (40%), Roma (IT)

(21) Appl. No.: 18/851,129

(22) PCT Filed: Mar. 24, 2023
(86) PCT No.: PCT/IB2023/052941

§ 371 Date: Sep. 26, 2024

(30) Foreign Application Priority Data
Mar. 30, 2022 (IT) 102022000006284

(51) Int. Cl. A61K 9/127

(52) U.S. Cl. CPC A61K 9/127

(57) ABSTRACT

The present invention relates to a method for the production of SUV, LUV and/or GUV unilamellar liposomes performed using chemical synthesis and an SDR (Spinning Disk Reactor) apparatus which together allow the production of SUV, LUV and GUV unilamellar liposomes, preferably with a controlled size; the method has a low energy consumption without the need for further post-treatment steps.

### FIELD OF THE INVENTION

The technical sector of the invention is that relating to the production of SUV, LUV and GUV unilamellar liposomes, preferably with a controlled or controllable size, namely size depending on the synthetic parameters at the start of the reaction, by means of specific apparatus, such as an SDR (Spinning Disk Reactor) apparatus.

### PRIOR ART

Liposomes are hollow spherical vesicles characterized by an inner aqueous core and an outer lipid bilayer, the structure of which makes them similar to human cell barriers (Patil and Jadhav, 2014). For this reason, liposomes may be used as drug or molecule carriers for dermatological, cosmetic, biomedical, therapeutic and nutraceutical purposes (Lian and Ho, 2001).

Unilamellar liposomes consist of a single phospholipid bilayer which encloses a hydrophilic core. Depending on their sizes, unilamellar liposomes may be further classified as follows (Ortega et al., 2017):
Nanometric small unilamellar liposomes (SUV), with a diameter varying between 1 nm and 100 nm;
Sub-micron large unilamellar liposomes (LUV) with a diameter varying between 100 nm and 1 μm;
Micrometric giant unilamellar liposomes (GUV), with a diameter greater than 1 μm.

The size of the unilamellar liposomes used is important for the possible action. For example, in the dermatological field, only SUV and LUV unilamellar liposomes with a diameter of up to 600 nm may easily pass through and beyond the stratum corneum (SC), the outermost layer of the epidermis, while the larger unilamellar liposomes tend to remain inside the SC (Bakonyi et al., 2018). On the other hand, smaller unilamellar liposomes with a diameter of 100 nm tend to pass beyond the entire epidermis and manage to reach the innermost layers, as far as the cardiocirculatory system, being dispersed inside the human body (Verma et al., 2003).

The production of SUV, LUV and GUV unilamellar liposomes may be performed using different techniques, including thin-film hydration, dialysis with detergents or reverse evaporation (Maja et al., 2020). However these techniques which are very widely used do not allow continuous operation, thus making it difficult to achieve production on an industrial scale. Costs are very high and this often results in the production of liposomes which are too large to be easily absorbed by the cells, with a substance trapping efficiency of less than 30%. These problems result in the need to add post-production steps in order to reduce the mean sizes of the particles and improve control of the particle size. This necessary choice often results in the loss of carried drug, with a consequent increase in costs and production time. Furthermore, discontinuous processes are not easily reproducible, with the presence of residual toxic solvent, owing to incompatibility with humans (Wagner et al., 2002).

For these reasons, the aforementioned techniques of the prior art may not be regarded as being low-cost, precise, fast, efficient, clean and continuous methods for the production of SUV, LUV and GUV unilamellar liposomes, preferably with a controlled size.

Other techniques used comprise the use of micromixers and spray dryers in a controlled environment or high-pressure systems. Even though these processes are characterized by the continuous production of sub-micron unilamellar liposomes and their size control is improved, the energy requirement of the apparatus used is very high-higher than 30,000 GJ per kg of sub-micron liposomes produced, considering a lipid concentration of 2 mg per ml of ethanol (Trucillo et al., 2020), respectively. It is emphasized that the technologies described here are unable to produce nanometric unilamellar liposomes.

Owing to their biodegradability, biocompatibility and size, SUV, LUV and GUV liposomes preferably with a controlled size are increasingly frequently used for the topical administration of active dermatological agents and in order to improve the stability of the formulations which contain easily degradable substances, including, for example, also antibiotics, proteins, DNA, vitamins, anti-tumour agents, stabilizers and anti-oxidants (Xie et al., 2020).

There therefore exists the need to provide a method for the synthesis and production of SUV, LUV and GUV liposomes, preferably with a controlled size, which is able to overcome the disadvantages or drawbacks of the prior art.

It is known to use spinning disk reactors (SDRs) for applications in technical sectors other than that for the production of SUV, LUV and GUV liposomes forming the subject of the present patent application.

It has been found that an SDR reactor provides advantages in the production of SUV, LUV and GUV liposomes, preferably with a controlled size.

### SUMMARY OF THE INVENTION

A first subject of the present invention is a method for the production of SUV, LUV and GUV unilamellar liposomes. preferably with a controlled size, performed using chemical synthesis and an SDR (Spinning Disk Reactor) apparatus.

The characteristic features obtained allow the production of SUV, LUV and GUV unilamellar liposomes, preferably with a controlled size, compatible with their use in the dermatological, cosmetic, biomedical, therapeutic and nutraceutical sector.

The present invention relates to a method, as described above, characterized in that it may be performed without the use of post-treatment steps.

The present invention relates to a method, as described above, where the SDR (Spinning Disk Reactor) apparatus is used for continuous production.

The present invention relates to a method, as described above, where the SDR (Spinning Disk Reactor) apparatus is used for a production method with an energy consumption which is limited to between 90 MJ and 450 MJ per kg of SUV, LUV and GUV unilamellar liposomes produced, preferably with a controlled size.

The present invention relates to a method, as described above, comprising the following steps:
preparation of a first aqueous solution A and a second solution B containing lipids dissolved preferably in an organic solvent;
activation of the SDR with rotation of the rotating disk;
injection of the reagent A in the centre of the disk for generation of a liquid film on the rotating disk;
injection of the solution B onto the surface of the disk, injected at a radial distance from the centre of the disk surface, in particular between 1 cm and 10 cm, preferably between 2 cm and 4 cm;
continuing rotation of the disk in order to cause the mixing of the reagent solutions in the liquid film and a continuous production of a reaction product of SUV, LUV and GUV liposomes;
collection of the reaction product. The organic solvent referred to in the first step is understood as being any organic solvent known in the art.

The present invention relates to a method, as described above, in which SDR reactor has at least one first nozzle for injection in the centre of the disk surface and at least one second nozzle for injection onto the disk surface at a radial distance from the centre of the disk surface, wherein preferably a distance between the injector nozzles and the disk surface is between 0.1 mm and 4 mm; preferably between 1 mm and 2 mm; and/or the said radial injection distance of one or more second injector nozzles is between 1 cm and 10 cm, preferably between 2 cm and 4 cm.

The present invention relates to a method, as described above, in which a speed of rotation of the disk of the SDR apparatus is set to between 200 rpm and 3000 rpm, preferably to between 300 and 1500 rpm.

The present invention relates to a method, as described above, in which a volumetric injection flowrate of the reaction solutions is between 10 ml/min and 1000 ml/min, preferably between 40 and 250 ml/min; and/or wherein the method is performed at ambient temperature and pressure.

The present invention relates to a method, as described above, characterized in that the SUV, LUV and GUV unilamellar liposomes obtained, preferably with a controlled size, are suitable for use in the dermatological, cosmetic, biomedical, therapeutic and nutraceutical sectors; the SUV, LUV and GUV unilamellar liposomes obtained, preferably with a controlled size, have:
a modal size of the SUV, LUV and GUV liposomes of between 1 and 200,000 nm, preferably 20-6000 nm;
a narrow and homogeneous, preferably monomodal, distribution, with a mean square deviation of between 5 and 50% with respect to the mean value, preferably of between 5 and 20% with respect to the mean value.

The present invention also relates to a method for the production of SUV, LUV and GUV unilamellar liposomes, preferably with a controlled or controllable size, for use as bioactive substance carriers in the dermatological, cosmetic, biomedical, therapeutic and nutraceutical sectors.

The SUV, LUV and GUV unilamellar liposomes, preferably with a controlled size, obtained using the method according to the present invention have preferably a modal size of between 1 and 200,000 nm, preferably 20-6000 nm; the modal size may be measured with a particle size analyzer, using conventional techniques.

Owing to their dimensions the controlled-size SUV, LUV and GUV unilamellar liposomes are particularly advantageous for dermatological, biomedical, pharmaceutical, therapeutic, cosmetic and nutraceutical uses; by way of a non-limiting example, it is envisaged using controlled-size SUV, LUV and GUV unilamellar liposomes as a vehicle for those substances with a biological or pharmacological effect which are not very soluble in aqueous solutions, increasing in fact the therapeutic availability thereof, also by means of the passage through biological membranes and fabrics. Such substances may include anti-biotics, proteins, DNA, vitamins, anti-tumour agents, stabilizers and anti-oxidants.

Further preferred characteristics of the SUV, LUV and GUV unilamellar liposomes, preferably with a controllable (predefined) size, according to the present invention comprise: a narrow and homogeneous, preferably monomodal, distribution, with a mean square deviation of between 5 and 50% with respect to the mean value, preferably of between 5 and 20% with respect to the mean value.

### BRIEF DESCRIPTION OF THE FIGURES

FIG. 1 shows the diagram of the SDR used.

FIG. 2 shows the size distribution of the SUV, LUV and GUV liposomes produced by means of the SDR operating at 576 rpm and with an EtOH/H₂O volume ratio of 0.5.

FIG. 3 shows the size distribution of the SUV, LUV and GUV liposomes produced by means of the SDR operating at 1440 rpm and with an EtOH/H₂O volume ratio of 0.2.

### DETAILED DESCRIPTION OF THE INVENTION

The method proposed in accordance with the present invention for the production of SUV, LUV and GUV liposomes, preferably with a controlled size, therefore has the advantage compared to the other systems of: (1) allowing the continuous production, compatible with industrial practice; (2) obtaining the product with an energy consumption, expressed in terms of kJ of energy used per kg of synthesis product, less than that of conventional methods; (3) obtaining a product with a controlled size within the nanometric to micron range; (4) decreasing the number of steps needed to obtain a synthesis product. These characteristics of the process overall differ from those commonly in use or of the prior art.

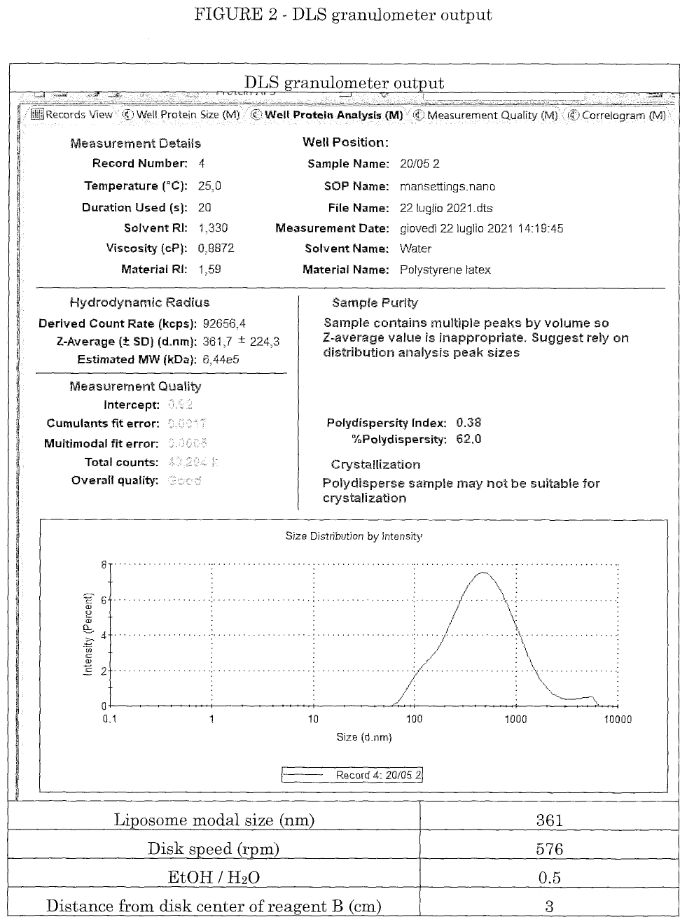

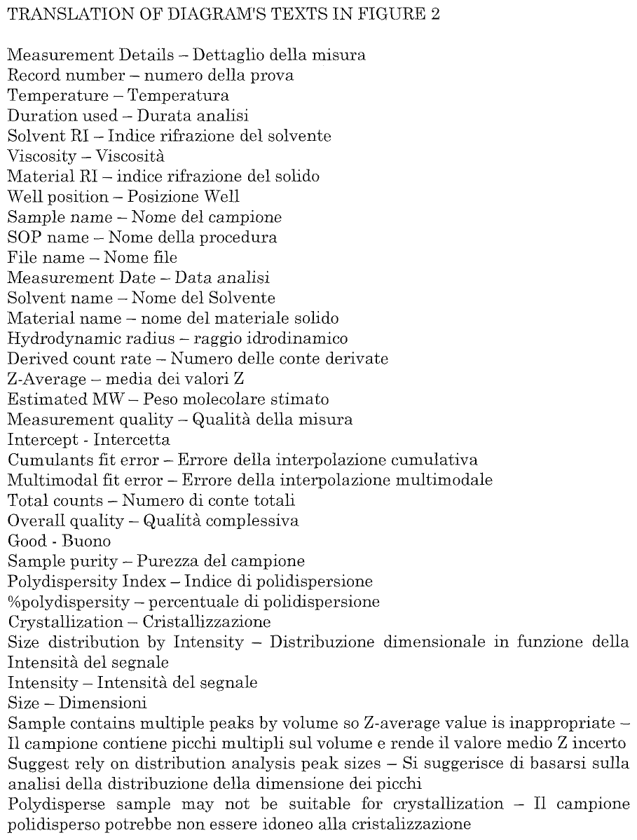

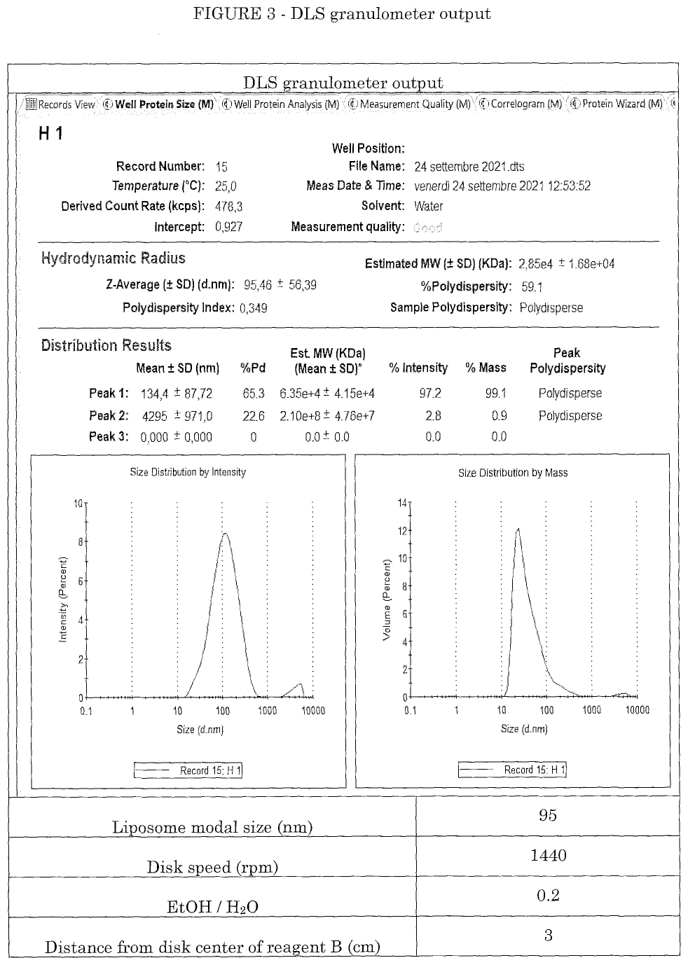

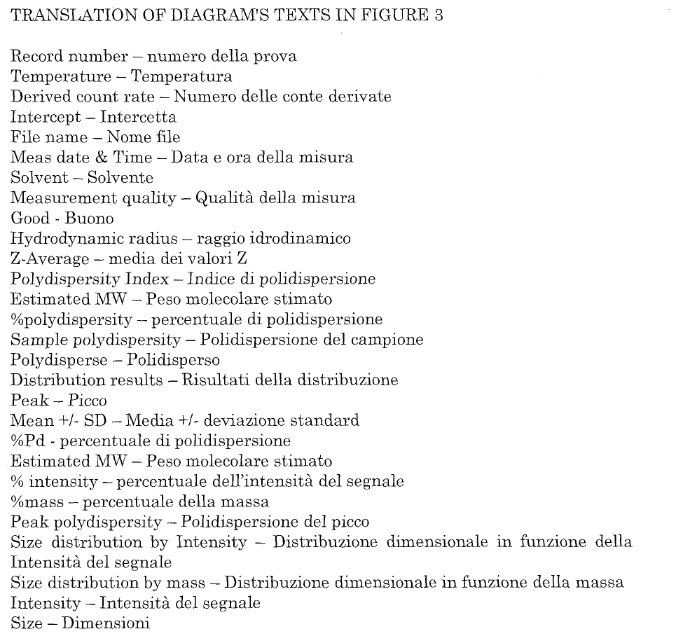

Experimentation is at an advanced level such as to justify the invention, with a proven production of SUV, LUV and GUV liposomes, preferably of controllable size, documented by particle size analyses (FIGS. 2 and 3).

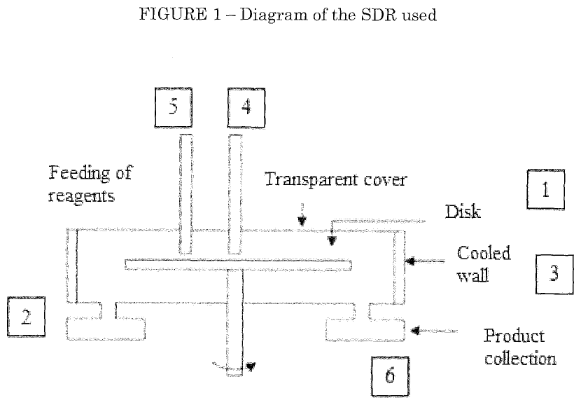

The production method which is proposed here involves the use of a spinning disk reactor (SDR). This type of reactor is known per se in the technology for applications in sectors other than the production of various SUV, LUV and GUV materials and, in a basic configuration shown in FIG. 1, comprises a rotating disk 1 with an upper surface suitable for the formation of a liquid film when the disk 1 is rotated by means of suitable operating means. The disk 1 is in particular inserted inside a chamber 2, the walls 3 of which are preferably cooled.

A plurality of injection nozzles 4, 5 are arranged to feed a liquid, in particular distilled water and/or reagent solutions, onto the surface of the disk. In particular, at least one first injection nozzle 4 is present in the centre of the disk and at least one second injection nozzle 5 is present on the surface of the disk at a given radial distance from the centre of the disk.

During use, the disk 1 is rotated at a process speed and, by means of injection of a reagent, a film of liquid is generated on the upper surface of the rotating disk 1. One or more reagent solutions are then injected onto the rotating disk 1 by means of the injection nozzles 4, 5; in particular, at least a first reagent solution is injected into the centre of the disk by means of the respective first nozzle 4 and at least one second reagent solution is injected at a radial distance from the centre of the disk by means of at least one respective second injection nozzle 5.

The rotation of the disk produces the necessary micromixing conditions in the liquid film so as to produce continuously SUV, LUV and GUV unilamellar liposomes, preferably with a controlled size.

The precipitated product is collected inside a container 6 arranged underneath the surface of the said disk. In particular, the product may precipitate tangentially with respect to the disk, striking the walls of the chamber and trickling down the walls towards the bottom, where an outlet for collection inside a container 6 is arranged.

The disk may have a diameter of between 5 and 100 cm, preferably 5 to 50 cm, for example 40 cm, and may be made of metallic, ceramic or polymer material, with or without a coating on the surface of the disk.

Suitable feeding means, such as injection pumps, may be provided in order to feed the reagents to the nozzles; a preferred volumetric flowrate for these feeding means is between 10 ml/min. and 1000 ml/min., preferably between 40 and 250 ml/min.

The process temperature and pressure may be monitored and/or controlled, if necessary, using methods known to the person skilled in the art, preferably at ambient temperature and pressure conditions.

A distance of the injection nozzles from the surface of the disk may be between 0.1 mm and 4 mm, preferably between 1 mm and 2 mm.

A radial distance between the point where injection is performed onto the surface of the disk by the one or more second injector nozzles (namely the vertical of nozzle) and the centre of the disk is preferably between 1 cm and 10 cm and is preferably between 2 cm and 5 cm.

For the synthesis reaction, a speed of rotation of the disk is preferably set to between 200 rpm and 3000 rpm, preferably between 300 rpm and 1500 rpm.

For the examples of the experimental tests described here, according to a preferred aspect, the SDR apparatus was equipped with a disk having a diameter of 40 cm and made of uncoated AISI 316 stainless steel.

### Example 1

The disk was driven at a rotational speed of 576 rpm by means of an electric motor connected to it. The reagent solutions consisted of a flow of distilled water (reagent A) and a 60% solution of ethanol and egg-yolk phosphatidylcholine (reagent B). These flows of reagents were simultaneously injected onto the surface of the disk by means of the injector nozzles, at the central point and at a distance of 3 cm from it, respectively. The product was collected in a container arranged underneath. The experiment was carried out injecting the reagents A and B both at a rate of 100 ml/min. The particles obtained were analysed by means of a Brookhaven 90 Plus particle size analyzer. The modal size of the controlled-size SUV, LUV and GUV unilamellar liposomes was 361 nm, with a narrow and homogeneous distribution as shown in FIG. 2.

### Example 2

The disk was driven at a rotational speed of 1440 rpm by means of an electric motor connected to it. The reagent solutions consisted of a flow of distilled water (reagent A) and a 60% solution of ethanol and egg-yolk phosphatidylcholine (reagent B). These flows of reagents were simultaneously injected onto the surface of the disk by means of the injector nozzles, at the central point and at a distance of 3 cm from it, respectively. The product was collected in a container arranged underneath. The experiment was carried out injecting the reagents A and B at a rate of 400 ml/min. and 100 ml/min., respectively. The particles obtained were analysed by means of a Brookhaven 90 Plus particle size analyzer. The modal size of the controlled-size SUV, LUV and GUV unilamellar liposomes was 95 nm, with a narrow and homogeneous distribution as shown in FIG. 3.

In general, the energy consumption in order to produce 12 g/h to 60 g/h of controlled-size SUV, LUV and GUV unilamellar liposomes is equal to 450 MJ and 90 MJ per kg of SUV, LUV and GUV unilamellar liposomes produced, preferably with a controlled size, respectively, and is much lower than that of other methods of the prior art.

### BIBLIOGRAPHY

Bakonyi, M., Gácsi, A., Berkó, S., Kovács, A., & Csányi, E. (2018). Stratum corneum lipid liposomes for investigating skin penetration enhancer effects. RSC advances, 8(48), 27464-27469.
Lian, T., & Ho, R. J. (2001). Trends and developments in liposome drug delivery systems. Journal of pharmaceutical sciences, 90(6), 667-680.
Maja, L., Željko, K., & Mateja, P. (2020). Sustainable technologies for liposome preparation. The Journal of Supercritical Fluids, 104984.
Ortega, V., Giorgio, S., & de Paula, E. (2017). Liposomal formulations in the pharmacological treatment of leishmaniasis: a review. Journal of liposome research, 27(3), 234-248.
Patil, Y. P., & Jadhav, S. (2014). Novel methods for liposome preparation. Chemistry and physics of lipids, 177, 8-18.
Trucillo, P., Campardelli, R., Iuorio, S., De Stefanis, P., & Reverchon, E. (2020). Economic Analysis of a New Business for Liposome Manufacturing Using a High-Pressure System. Processes, 8(12), 1604.
Verma, D. D., Verma, S., Blume, G., & Fahr, A. (2003). Particle size of liposomes influences dermal delivery of substances into skin. International journal of pharmaceutics, 258(1-2), 141-151.
Wagner, A., Vorauer-Uhl, K., & Katinger, H. (2002). Liposomes produced in a pilot scale: production, purification and efficiency aspects. European journal of pharmaceutics and biopharmaceutics, 54(2), 213-219.
Xie, P., Dolivo, D. M., Jia, S., Cheng, X., Salcido, J., Galiano, R. D., . . . & Mustoe, T. A. (2020). Liposome-encapsulated statins reduce hypertrophic scarring through topical application. Wound Repair and Regeneration, 28(4), 460-469.

1. Method for the production of (SUV) nanometric small unilamellar liposomes, (LUV) sub-micron large unilamellar liposomes, and (GUV) micrometric giant unilamellar liposomes, said method comprising
performing chemical synthesis and using an SDR (Spinning Disk Reactor) apparatus.

2. Method according to claim 1, wherein said method is performed without post-treatment processes.

3. Method according to claim 1, wherein the SDR apparatus is used for continuous production.

4. Method according to claim 1, wherein the SDR apparatus is used for a production method with an energy consumption which is limited to between 90 MJ and 450 MJ per kg of controllable-size SUV, LUV and GUV unilamellar liposomes produced.

5. Method according to claim 1, comprising the following steps:
preparing a first aqueous solution A and a second solution B containing lipids dissolved in an organic solvent;
activating the SDR with rotation of a rotating disk;
injecting the first aqueous solution A centrally in the rotating disk for generation of a liquid film on the rotating disk;
injecting the second solution B onto the disk surface at a radial distance from the disk surface center;
continuing rotating of the disk in order to cause mixing of the first aqueous solution A and of second solution B in the liquid film and continuously producing a reaction product of SUV, LUV and GUV liposomes; and
collecting the reaction product.

6. Method according to claim 5, wherein the SDR apparatus has at least one first nozzle for injection in the center of the disk surface and at least one second nozzle for injection onto the disk surface at a radial distance from the center of the disk surface, wherein a distance between the injector nozzles and the disk surface is between 0.1 mm and 4 mm; and/or the said radial injection distance of one or more second injector nozzles is between 1 cm and 10 cm.

7. Method according to claim 6, wherein a speed of rotation of the disk of the SDR apparatus is set to between 200 rpm and 3000 rpm.

8. Method according to claim 5, wherein a volumetric injection flowrate of the reagent solutions is between 10 ml/min. and 1,000 ml/min.; and/or wherein the method is performed at ambient pressure and temperature.

9. Method for the production of SUV, LUV and GUV unilamellar liposomes according to claim 1, wherein the controllable-size SUV, LUV and GUV unilamellar liposomes obtained are suitable for use in the dermatological, cosmetic, biomedical, therapeutic and nutraceutical sectors; the controlled-size SUV, LUV and GUV unilamellar liposomes obtained have:
a modal size of the SUV, LUV and GUV liposomes of between 1 and 200,000 nm;
a narrow and homogeneous distribution, with a mean square deviation of between 5 and 50% with respect to the mean value.

10. Method for the production of SUV, LUV and GUV liposomes according to claim 1, as bioactive substance carriers in the dermatological, cosmetic, biomedical, therapeutic and nutraceutical sectors.

11. The method according to claim 1, wherein said SUV, LUV and GUV unilamellar liposomes have a controlled size.

12. The method according to claim 5, wherein said radial distance from the disk surface center is between 1 cm and 10 cm.

13. The method according to claim 5, wherein said radial distance from the disk surface center is between 2 cm and 4 cm.

14. The method according to claim 6, wherein the distance between the injector nozzles and the disk surface is between 1 mm and 2 mm.

15. The method according to claim 6, the said radial injection distance of one or more second injector nozzles is between 2 cm and 4 cm.

16. The method according to 7, wherein the speed of rotation of the disk of the SDR apparatus is set to between 300 rpm and 1500 rpm.

17. The method according to claim 8, wherein the volumetric injection flowrate of the reagent solutions is between 40 and 250 ml/min.

18. The method according to claim 9, wherein the modal size of SUV, LUV and GUV liposomes is 20-6000 nm.

19. The method according to claim 9, wherein the narrow and homogeneous distribution has a mean square deviation of between 5 and 20% with respect to the mean value.

20. The method according to claim 9, wherein said SUV, LUV and GUV liposomes have a monomodal distribution.
